(19) United States
(12) Patent Application Publication
(10) Pub. No.: US 2004/0241259 A1
(43) Pub. Date: Dec. 2, 2004

(54) Herbal ointment and method for constipation treatment

(76) Inventor: Christina Lim, Singapore (SG)

Correspondence Address: Donald W. Meeker, Patent Agent, 924 East Ocean Front #E, Newport Beach, CA, 92661, US

(21) Appl. No.: 10/448,012

(22) Filed: May 29, 2003

(51) Int. Cl. A61K 35/78

(52) U.S. Cl. 424/757; 424/773; 424/775

(57) ABSTRACT

A topical treatment for functional constipation in humans, wherein active herb encouraging the peristaltic movement of the stomach and the intestines, increasing water secretion is invented by applying an herbal topical preparation inside and/or around the navel. The treatment results in a significant non-invasive relief of functional constipation without discomfort.

### BACKGROUND OF THE INVENTION

1. Field of the Invention

This invention relates to topical pharmaceutical preparation and more particularly to the filed of constipation relief by the use of topically applied agents including at least one of the herbs Cortex Moutan, Lignum Aquilariae Resinatum, Radix Angelicae Dahuricae and Bomedlum Synthethicum.

2. Description of the Prior Art

Herbal medicines or herbal drugs are of ancient origin and their use is known in cultures throughout the world. Chinese herbal medicine is among the earliest known examples of organized scientific study and formulation of herbal treatments. Herbalism is known to have been popular in China as early as 2500 BC.

More than 5% of apparently healthy people complain of constipation and regularly use laxatives. However, chronic laxative consumption orally or anally may damage the colonic muscle layer and lead to a true colonic cause of constipation. Thus, a vicious cycle is initiated. Other medications like gastrokinetic or enterokinetic agents stimulate the motility of gastrointestinal tract by releasing acetylcholine (ACE) from the nerve endings of the myenteric plexus. Although these medications are effective, but side effects like nausea, dyspepsia and anxiety have been observed in patients taking these medications.

Prior art U.S. Pat. No. 5,910,308, issued Jun. 6, 1999 to D'Jang, provides an herbal extract-based composition comprising an extract of Gynostemma pentaphyllum, an extract of Crataegus pinnatifida (hawthorn leaves or berries), and an extract of Camellia sinensis (green tea). Also provided is a process for preparing a herbal extract-based composition which comprises separately extracting each of hawthorn berries, green tea leaves, and Gynostemma pentaphyllum leaves; drying extraction eluates obtained from the extracting of each of hawthorn berries, green tea leaves, and Gynostemma pentaphyllum leaves to obtain organic residues in forming a hawthorn berry extract powder, green tea extract powder, and a Gynostemma pentaphyllum extract powder; and combining the green tea extract powder, the Gynostemma pentaphyllum extract powder, and the hawthorn berry extract powder in desired proportions to form the herbal extract-based composition which has health promoting effects including potent inhibition of free radicals.

Prior art U.S. Pat. No. 121,904, issued Dec. 12, 1871 to Simmons, provides an improvement in medical compounds for treating constipation. To prepare this medicine, take six ounces of senna, three ounces of serpentaria Virginia, two ounces of apocynum androsoemifolium, and two ounces of buchu. Put these ingredients in an earthenware vessel and pour upon them a gallon of boiling water. Cover the mixture so as to prevent the escape of steam and to allow it to stand twenty-four hours. Then add half a gallon of proof spirits and stir the mixture thoroughly. Recover and allow it to stand twenty-four hours longer. Strain well through a linen or cotton cloth. When cool, the medicine is ready for use.

Prior art U.S. Pat. No. 2,938,832, issued May, 31, 1960 to Huggins, shows an improved laxative composition adapted to effect elimination of hardened or impacted feces with a minimum of pain and discomfort, by using a nontoxic salt of a dialkyl sulfosuccinate.

Prior art U.S. Pat. No. 6,416,795, issued Jul. 9, 2002 to Choi, claims antistress compositions containing various medicinal herbs which are composed of one or more medicinal herbs. 2.0-40.0 g of fructus hordei germintus, messa medicata fermentata, fructus amomi and fructus crataegei; one or more medicinal herbs 2.0-40.0 g of radix polygalae, semen zizyphi spinosae and rhizoma acori graminei; one or more medicinal herbs 2.0-40.0 g selected from the group consisting of poria holen and poria, one or more medicinal herbs 2.0-40.0 g of folium phyllostachys, calus in tacin phyllostachys, succus phyllostachys and radix scutellariae; one or more medicinal herbs 2.0-40.0 g of germinated Glycine max (L.) Merr, fructus amomicardamomi, semen glycine and semen lablab; one or more medicinal herbs 2.0-40.0 g of rhizoma zingiberis, radix asari, rhizoma zingiberis siccatum and radix aconiti; one or more medicinal herbs 2.0-40.0 g of bulbos allii fistulosi, herba ephedrae and folium perillae; and one or more medicinal herbs 2.0-40.0 g of red pepper and radix platycodi as the effective ingredients that relieve stress and repress memory failure.

Prior art U.S. Pat. No. 6,428,822, issued Aug. 8, 2002 to Shi, describes a mixed substance for treating hypertension, constipation, detoxification, and boosting the immune system produced by extracting arctium lappa L., carrot, and whole radish with water one or two hours at temperature 70.degree. C. 100. degree. C. under agitating, separated the extracts and solid by-products, vacuum condensed the extracts, then at low temperature lyophilized condensed extracts to powder, encapsulated powder or pressed powder to tablet. Patients taking a daily dosage of this vegetable medicine have shown greatly improved healthy condition.

Prior art U.S. Pat. No. 6,083,507, issued Jul. 4, 2000 to Belle, discloses a composition for eliminating the buildup of harmful toxins and improving energy, mental clarity and waste elimination. The composition comprises quantities of gotu-kola, whole leaf aloe vera, lactobacilli organism and folic acid.

Prior art U.S. Pat. No. 6,060,063, issued May 9, 2000 to Lansky, indicates a phytoestrogen supplement and the methods of its preparation. The basis of the oral phytoestrogen supplement was a preparation of the pomegranate material, preferably pomegranate seeds, which were contacted with an appropriate solvent as described below, preferably including water and ethanol. Preferably, the supplement also included an herbal mixture, including schizandra berries and Chinese asparagus root, and most preferably including Chinese licorice root and Chinese angelica root. The phytoestrogen supplement can also be prepared from pomegranate material alone without the addition of other herbals, which involves fermenting the pomegranate material including the pomegranate seeds. The basis of topical phytoestrogen supplements was a mixture of a preparation of the pomegranate material, preferably pomegranate seeds, which were pressed as described below to produce pomegranate oil, and of the coconut milk. Preferably, the supplements also included schizandra berries and Chinese asparagus root.

Prior art U.S. Pat. No. 4,809,370, issued Mar. 7, 1989 to Juan, puts forth an apparatus for assisting in the evacuation of the waste matter (feces) present in the rectum and sigmoid colon. The apparatus consists of a frame-like structure supporting a container of warm water so positioned that the anus of the user is submersed in the water. Adapted for use on a flush toilet, connections are included to a source of warm water. Also included as a part of the water container are means for emptying the container into the associated toilet while the apparatus is in use.

Prior art U.S. Pat. No. 4,670,920, issued Jun. 9, 1987 to Juan, concerns an apparatus for assisting in the evacuation of the bowel and sigmoid colon. The apparatus consists of a frame-like structure supporting a container of warm water so positioned that the anus of the user is submersed in the water. Adapted for use on a flush toilet, connections are included to a source of warm water. Also included as a part of the water container are means for emptying the container into the associated toilet while the apparatus is in use.

Prior art U.S. Pat. No. 6,280,751, issued Aug 28, 2001 to Fletcher, illustrates new medicinal and cosmetic compositions that comprise essential oils in combination with herbs and/or spices. The compositions may be used orally or topically.

Prior art U.S. Pat. No. 6,193,995, issued Feb. 27, 2001 to Zhenzhen, is for an herbal mixture to treat a user for nasal congestion. The herbal mixture comprises a plurality of herbs thoroughly mixed together and combined with an adhesive. The plurality of herbs comprise Fructus Piperis nigri herb, Rhizoma Zingiberis officinalis herb, and Ramulus Cinnamomi Cassiae herb. The Fructus Piperis nigri herb is in a range from 0.1% to 99.8%. The Fructus Piperis nigri herb is preferably 33.3%. The Rhizoma Zingiberis officinalis herb is in a range from 0.1% to 99.8%. The Rhizoma Zingiberis officinalis herb is preferably 33.3%. The Ramulus Cinnamomi Cassiae herb is in a range from 0.1% to 99.8%. The Ramulus Cinnamomi Cassiae herb is preferably 33.3%.

Prior art U.S. Pat. No. 5,162,037, issued Nov. 10, 1992 to Whitson-Fischman, provides a method for treating pathogenic conditions of the human body by preparing a homeopathic mixture of at least one herb, herbal extract or other compound exhibiting therapeutic properties, adding a magnetically permeable substance to the mixture if necessary, magnetizing the resulting mixture to impart a substantially unipolar magnetic charge on the mixture and administering the magnetized mixture through one or more specific acupuncture points associated with producing a desired response to the particular condition being treated. The invention is also directed to the treatment of various diseases through the oral, auricular, topical or injectable administration of magnetically influenced homeopathic medicaments.

Prior art U.S. Pat. No. 6,048,533, issued Apr. 11, 2000 to Nguyen, shows the administration of an effective amount of turmeric for the treatment of various health ailments and also as a food supplement to promote health and vitality. The turmeric is especially useful for the treatment of skin disorders, such as acne, when administered orally. It can also be applied topically as a whitened or bleached composition. It can also be used to treat liver and stomach disorders, skin discoloration, constipation, and hemorrhoids

What is needed is a method of utilizing the topical application of an herbal ointment/other topical preparation inside and/or around the navel to achieve a relief of constipation without any discomfort.

### SUMMARY OF THE INVENTION

The primary object of the present invention is to provide a topically applied herbal ointment or other topical preparation and a method of utilizing the topical application of the herbal ointment/other topical preparation inside and/or around the navel to achieve a relief of constipation without any discomfort.

In brief, the herbal ointment/topical preparation is a combination of herbal extracts, which relieve pain, stimulate blood and Qi (energy) circulations, enhance the function of the stomach and the intestines. Each of the herbs has different effects through different action pathway/pathways, the herbs comprising any single herb or combination of herbs taken from the list of herbs including Cortex Moutan, Lignum Aquilariae Resinatum, Radix Angelicae Dahuricae and Bomedlum Synthethicum.

The center of navel is an acupuncture point in Traditional Chinese Medicine (TCM). It is known as ShenQue6 and is believed to posses a very important role in gastrointestinal function and well-being. However, in this invention, the herbal ointment is applied inside and/or around the navel based on the following facts: (1) the active herbs enhance the gastrointestinal function, as well as the general health by stimulating the acupuncture point, “ShenQue”; (2) the skin of the navel is the thinnest part in the body. The herbs in the ointment penetrate the skin better and act on the stomach, small and large intestines through the portal vine circulation; (3) this method is safe as very little active herbs go in the main blood circulation.

This invention has many advantages over existing methods and medications for functional constipation in that it is easy to apply, safe, painless and effective in the relief of constipation, as well as improving gastrointestinal function.

### BEST MODE FOR CARRYING OUT THE INVENTION

A topical ointment or other topical preparation for effective relief of constipation without any discomfort, the ointment comprising an herbal ointment or other topical preparation comprising a base mixture capable of topical application on the human skin inside of the navel and/or around the navel and at least one of the herbs selected from the list of herbs including Cortex Moutan, Lignum Aquilariae Resinatum, Radix Angelicae Dahuricae and Bomedlum Synthethicum.

The topical ointment may comprise the base mixture capable of topical application on the human skin inside of the navel and/or around the navel and one, two, three, or all four of the herbs selected from the list of herbs including Cortex Moutan, Lignum Aquilariae Resinatum, Radix Angelicae Dahuricae and Bornedlum Synthethicum.

In practice, a non-invasive method for effective relief of constipation without any discomfort, the method comprises a first step of preparing an herbal ointment or other topical preparation having a base mixture capable of topical application on the human skin and at least one of the herbs selected from the list of herbs including Cortex Moutan, Lignum Aquilariae Resinatum, Radix Angelicae Dahuricae and Bornedlum Synthethicum and a second step of applying the herbal ointment to the skin of a human inside of the navel and/or around the navel.

The method may comprise preparing the herbal ointment having a base mixture capable of topical application on the human skin and one, two, three, or all four of the herbs selected from the list of herbs including Cortex Moutan, Lignum Aquilariae Resinatum, Radix Angelicae Dahuricae and Bomedlum Synthethicum.

Treatment of Constipation with Ocean Health Aculax™, the trademarked name for the herbal ointment of the present invention, was observed in the following clinical study.

Summary of First Clinical Study

A 4-month clinical trial was carried out to investigate the effects of Aculax™, a Chinese herbal ointment, on constipation in 100 outpatients. The 100 patients diagnosed with functional constipation (age 18-65) were given Aculax™ 0.5-1 g, 2-3 times daily, applied inside the navel with a firm massage in circular motion. The following results observed: (1) Relief of constipation was observed in 98 out of 100 patients; (2) Fifty-three out of the 100 patients recovered from functional constipation after applying Aculax™ for a few times; (3) An immediate relief was observed in 6% of the patients; (4) 30% of the patients felt the effect within 3 hours; (5) Up to 77% of the patients experienced the relief within 24 hours; while another 21% of the patients got relief within 48 hours. It is concluded that Aculax™ is a safe and effective relief of functional constipation.

Methods

Patient Inclusion Criteria

In the present study, constipation was defined as a condition, which the interval of bowel movement was 72 and more than 72 hours, accompanied with dry hardened or pellet stool or difficult evacuation of the stool.

In Traditional Chinese Medicine (TCM), constipation is considered as a disorder of Qi (energy) stagnation with one or more of the following signs and symptoms: difficult evacuation of the stool, prolonged intension of bowel movement after evacuation, dry hardened stool, bloating, frequent belching, bowstring pulse, yellow tongue fur. And this disorder is closely related to mood swings.

Patients falling in the above-mentioned criteria were encouraged to enter this trial unless one or more of the following exclusion criteria were fulfilled: (1) Narrow intestinal tract caused by organic diseases in the colon and rectum including tumor, polyp and tuberculosis. (2) Patients aged below 18 years old and above 65 years old; pregnant women and nursing mothers; patients who are allergic to Aculax™. (3) Patients with severe cardiovascular, hepatic, renal, hemopoietic and mental diseases. (4) Patients with incomplete data in this trial were excluded for result evaluation.

Patients

42 male and 58 female patients were involved in this trial. The age range of the study population was 18-65 years old; patients with functional constipation made up 37 cases, while constipated patients with other complications made up 63 cases including 21 diabetics, 15 patients with urethritis, 8 patients with chest pain, 7 insomnia patients, 5 patients with flu, 4 stroke patients and 3 patients with vertigo. Distributions of the age and the duration of constipation in the study population were given below in Table 1.

TABLE 1
Distributions of the age and the duration of constipation
in the study population

| Range of | No. of | Duration of | | No. of |
|---|---|---|---|---|
| age | Patients | Constipation | | Patients |
| 18-30 | 29 | 1 | month | 76 |
| | | 1-3 | months | 3 |
| 30-50 | 49 | 3-6 | months | 5 |
| | | 6-12 | months | 9 |
| 50-65 | 22 | 1 | year | 7 |

Treatment

Aculax™ was applied inside the navel with a firm massage for 30 seconds, 0.5-1 g, 2-3 times a day until constipation relief was achieved. The treatment course lasted 1-2 days, and could be repeated for 3-5 times.

Parameters Monitored

Safety Parameters

A. general medical check-up;

B. normal blood, urine and stool tests;

C. heart, liver and kidney function tests.

Efficacy Parameters

A. stool frequency, stool consistency and difficulty in bowel movement;

B. normal stool test;

C. barium enema examination;

D. fiber-colon scope examination. Items A and B in 4.1 and 4.2 were compulsory, the rest was optional in present study.

Result Evaluation Criteria

Five clinical grades of constipation were classified as −, ±, +, ++ and +++, which indicated the following conditions respectively: mild constipation occurred sometimes; mild constipation occurred at a regular basis; constipation occurred at a regular basis; sustained constipation; and sustained constipation resulting in medical consultation.

In the current study, there were five outcomes observed as follows:

Clinical recovery (CR): Bowel movement became normal without any other symptoms.

Significant improvement (SI): bowel movement interval was within 72 hours with mild symptoms below: dry but close to normal stool, mild bloating etc.

Improvement (I): the interval of bowel movement shortened for 1 day after the treatment or stool consistency improved with relief from the accompanied signs and symptoms.

No improvement (NI): No improvement observed in constipation or the accompanied signs and symptoms.

Results

TABLE 2
General effects

Evaluation Criteria

| | CR | SI | I | NI |
|---|---|---|---|---|
| No. of patients | 53 | 27 | 18 | 2 |

Notes:
The clinical recovery rate was 53% and the general effective rate was 98%.

TABLE 3
The time from Aculax ™ application to the relief
of constipation achieved

| | Time | | | | | | |
|---|---|---|---|---|---|---|---|
| | | | | | 6-12 | 12-24 | 24-48 |
| | Immediate | 1 hr | 1-3 hrs | 3-6 hrs | hrs | hrs | hrs |
| Patients | 6 | 12 | 12 | 14 | 16 | 17 | 23 |

Notes:
18% of patients achieved relief of constipation within 1 hour; 30% of patients achieved relief of constipation within 3 hours; 77% of patients achieved relief of constipation within 24 hours. This result indicates that Aculax ™ is an effective and fast onset herbal ointment for constipated patients.

TABLE 4
Comparison on symptoms of constipation before and after Aculax ™
treatment

| | Before Treatment | | | | | After Treatment | | | | | | |
|---|---|---|---|---|---|---|---|---|---|---|---|---|
| Symptoms | +++ | ++ | + | +/− | − | +++ | ++ | + | +/− | − | μ | P |
| Difficult | 9 | 44 | 47 | 0 | 0 | 0 | 1 | 8 | 5 | 84 | 37.124 | <0.01 |
| evacuation of | | | | | | | | | | | | |
| the stool | | | | | | | | | | | | |
| Prolonged | 0 | 19 | 77 | 0 | 4 | 0 | 0 | 9 | 3 | 88 | 36.190 | <0.01 |
| intension of | | | | | | | | | | | | |
| bowel | | | | | | | | | | | | |
| movement | | | | | | | | | | | | |
| Bloating | 0 | 7 | 81 | 2 | 10 | 0 | 0 | 3 | 4 | 93 | 35.235 | <0.01 |
| Fullness and | 0 | 9 | 82 | 4 | 5 | 0 | 0 | 1 | 3 | 96 | 38.309 | <0.01 |
| pain in | | | | | | | | | | | | |
| The abdomen | | | | | | | | | | | | |
| Frequent | 0 | 3 | 65 | 9 | 23 | 0 | 0 | 1 | 4 | 95 | 30.50 | <0.01 |
| Belching | | | | | | | | | | | | |
| Close relation | 0 | 27 | 59 | 4 | 19 | 0 | 0 | 12 | 7 | 81 | 28.652 | <0.01 |
| with | | | | | | | | | | | | |
| Mood swings | | | | | | | | | | | | |

Notes:
The above result of self-comparison showed that Aculax ™ x improved most of the accompanied symptoms of constipation significantly (P < 0.01).

TABLE 5
Comparison on tongue texture and fur of patients before and
after Aculax ™ treatment

| | | |
|---|---|---|
| | Before | After |
| Tongue | treatment | treatment |

| | | | |
|---|---|---|---|
| Tongue | light red | 71 | 80 |
| Texture | red | 18 | 18 |
| | dark red | 8 | 0 |
| | light gray | 3 | 2 |

| | |
|---|---|
| X_{—} | 7.4 |
| p | <0.05 |

| | | | |
|---|---|---|---|
| Tongue | white & thin | 69 | 95 |
| Fur | white & wet | 25 | 5 |
| | yellow | 6 | 0 |

| | |
|---|---|
| X_{—} | 245 |
| p | <0.01 |

Note:
Significant improvements on tongue texture and fur were observed after the treatment (p < 0.05).

TABLE 6
Comparison on pulse before and after Aculax ™ treatment

| | Before | After |
|---|---|---|
| Pulse | treatment | treatment |
| string & forceful | 43 | 4 |
| taut & slippery | 9 | 3 |
| deep & weak | 22 | 1 |
| soft & slow | 14 | 35 |
| slow | 12 | 57 |

| | |
|---|---|
| X_{—} | 91.84 |
| p | <0.01 |

Note:
A significant improvement on pulse was observed after the treatment (p < 0.05).

TABLE 7
The relationship between the treatment outcome of constipation
and patient's age

| | Age | | |
|---|---|---|---|
| | 18-30 | 30-50 | 50-65 |
| No. of patients | 29 | 49 | 22 |
| CR | 16 | 27 | 10 |
| SI | 10 | 12 | 5 |
| I | 3 | 9 | 6 |
| NI | 0 | 1 | 1 |
| R | 0.4676 | 0.4925 | 0.5593 |

| | |
|---|---|
| X_{—} | 1.3195 |
| P | >0.05 |

Note.
There was no significant relationship between the treatment outcome and patient's age, X² = 1/3195, p < 0.05.

TABLE 8
The relationship between the treatment outcome of constipation
and the duration of constipation

The duration of constipation

| | 1 | 1-3 | 3-6 | 6-12 | >1 |
|---|---|---|---|---|---|
| | month | months | months | months | year |
| No. of | 76 | 3 | 5 | 9 | 7 |
| patients | | | | | |
| CR | 46 | 1 | 2 | 2 | 2 |
| SI | 18 | 2 | 2 | 2 | 2 |
| I | 11 | 0 | 1 | 3 | 3 |
| NI | 1 | 0 | 0 | 1 | 0 |
| R | 0.4597 | 0.5317 | 0.55 | 0.6872 | 0.69 |

| | |
|---|---|
| X_{—} | 8.484 |
| P | >0.05 |
| | <9.488 |

Note:
There was no significant relationship between the treatment outcome and the duration of constipation (X² = 8.848, p < 0.05).

Conclusions

Aculax™ is a Chinese herbal medicine processed by advanced technology. This study shows that it is an effective and quick relief for constipation; There were no abnormal changes of safety parameters measured in this study. Therefore, Aculax™ is safe.

In the present study, significant improvements were observed in constipation and its accompanied symptoms, tongue texture, tongue fur and pulse. This indicates that Aculax™ not only regulates the bowel movement, but also enhances the functions of gastrointestinal system. Age and the duration of constipation did not affect the efficacy of Aculax™ in this study.

This study indicates that Aculax™ is effective for functional constipation or constipation caused by Qi (energy) stagnation. However, further studies need to be done to see whether Aculax™ works on constipation caused by systemic diseases. Notes: In TCM, normal pulse should be strong with moderate pace (70-90/min); normal tongue texture is light red and tongue fur is white and thin.

Summary of Second Clinical Study

Treatment of Chronic Constipation with Aculax™ and Placebo

Patients

Patients with chronic constipation¹ (less than 3 spontaneous bowel movements a week) and constipated patients reporting regular laxative consumption of at least 1 year duration were qualified to enter the base line phase unless one or more of the following exclusion criteria were present:

(1) intermittent diarrhea;

(2) organic diseases of the colon such as tumors and polyp;

(3) acute or chronic inflammatory diseases;

(4) organic diseases of the rectum and anus such as fissures, fistulas, perianal abscesses, and eczema of the perianal skin;

(5) regular intake of antacids or of opioid, anticholinergic, anticonvulsant, antidepressant or neuroleptic drugs;

(6) hypothyroidism or hyperparathyroidism;

(7) malignant diseases;

(8) pregnancy

(9) patients underwent gastrointestinal surgery within one week before the trial. 30 patients (aged from 20-80 years old) with chronic constipation entered this trial. 24 of them were women; 8 (33%) of them regularly used laxatives such as Ducolax, Bekunis, Senna and other herbal tea.

Study Design

The study lasted for 10 weeks and consisted of three phases. The first phase was called the base line phase and lasted for 2 weeks. It was followed by a four-week treatment phase during which all patients were given Aculax™. Finally, a placebo was given to all patients for 4 further weeks (run out phase). Studies¹ have shown that 2 weeks was needed to build up a normal bowel habit. A 4-week period is an effective time period to observe Aculax™ or placebo's effects on bowel movement habit.

Patients were required to record their daily food, laxative and fiber supplement intakes, bowel movement by using a diary booklet, and were seen at the end of week 2, 4, 6 and 8. At each visit, the patient assessed his situation with respect to constipation by means of a visual analogue scale². A horizontal line of 100 mm length was labeled at its left end “as bad as always”, at its right end “could not be better”. Meanwhile, blood pressure, heart rate, and body weight were registered and the patient was asked for possible side effects of treatment. The diary booklets were checked for completeness. Patients suspected of not having completed the booklet properly were excluded from the study.

The Purpose of the Study on AcuLax™:

(1) Investigate effects of AcuLax™ on bowel habit.

(2) The capability of AcuLax™ in reducing laxative intake in patients with chronic laxative abuse

Results

Treatment was well-tolerated in Aculax™ and placebo phase. No change in blood pressure, heart rate and body weight were observed. The following side effects were reported: rashes and pulling fell around the navel, nausea, from both Aculax™ and placebo groups. Three patients refused to continue because of lack of effects in the aged patients (70 years old and above). One patient on Aculax™ and placebo each dropped out because of skin rashes. One patient was withdrawn due to incomplete data.

Total stool frequency was significantly (p<0.05) increased from 5.79±2.64 per 2 weeks to 8.38±3.68 per 2 weeks by Aculax™. Furthermore, this effect was maintained for about one month during the wash-out phase. Please refer to FIG. 1. Frequency of Bowel Movement.

[image]

Subjective Assessment on Constipation Status

Aculax™ treatment was considered to be effective by 22 out of 24 patients, who completed the study. The effective rate was about 91.7% (22/24) as assessed by the visual analogue scale. Please refer to FIG. 2:

[image]

Laxative Consumption in the 8 Participants.

After one-month treatment with Aculax™, 6 out of 8 participants (75%) were able to stop using laxatives and one cut down the use of laxative from 3 times a week to once a week. One failed to see any improvement, as the regular consumption of laxative could weaken the muscle of the intestines.

Conclusion

The results suggest that Aculax™ is a safe and an effective herbal medicine for chronic constipation. It relieves constipation, also helps in building up a healthy bowel movement habit after one-month treatment. This reflects from the increased frequency of bowel movement, a higher subjective assessment score and the reduced dosage or complete cutting-down of laxatives in the wash out phase in present study.

References

1. S. A. Muller-Lissner and The Bavarian Constipation Study Group Treatment of chronic constipation with cisapride and placebo Gut, 1987, 28, 1033-1038

2. Alyson Bone and Malcolm Lader The use of analogue scales in rating subjective feelings Br J Med Psychol 1974, 47, 211-218

It is understood that the preceding description is given merely by way of illustration and not in limitation of the invention and that various modifications may be made thereto without departing from the spirit of the invention as claimed.

What is claimed is:

1. A non-invasive method for effective relief of constipation without any discomfort, the method comprising:
a first step of preparing a topical preparation having a base mixture capable of topical application on the human skin and at least one of the herbs selected from the list of herbs including Cortex Moutan, Lignum Aquilariae Resinatum, Radix Angelicae Dahuricae and Bomedlum Synthethicum;
a second step of applying the topical preparation to the skin of a human inside of the navel and/or around the navel.

2. The method of claim 1 wherein the first step comprises preparing the topical preparation having a base mixture capable of topical application on the human skin and one of the herbs selected from the list of herbs including Cortex Moutan, Lignum Aquilariae Resinatum, Radix Angelicae Dahuricae and Bornedlum Synthethicum.

3. The method of claim 1 wherein the first step comprises preparing the topical preparation having a base mixture capable of topical application on the human skin and two of the herbs selected from the list of herbs including Cortex Moutan, Lignum Aquilariae Resinatum, Radix Angelicae Dahuricae and Bomedlum Synthethicum.

4. The method of claim 1 wherein the first step comprises preparing the topical preparation having a base mixture capable of topical application on the human skin and three of the herbs selected from the list of herbs including Cortex Moutan, Lignum Aquilariae Resinatum, Radix Angelicae Dahuricae and Bomedlum Synthethicum.

5. The method of claim 1 wherein the first step comprises preparing the topical preparation having a base mixture capable of topical application on the human skin and all four of the herbs selected from the list of herbs including Cortex Moutan, Lignum Aquilariae Resinatum, Radix Angelicae Dahuricae and Bomedlum Synthethicum.

6. The method of claim 1 wherein the topical preparation is an herbal ointment.

7. A topical preparation for effective relief of constipation without any discomfort, the topical preparation comprising:
a topical mixture comprising a base mixture capable of topical application on the human skin inside of the navel and/or around the navel and at least one of the herbs selected from the list of herbs including Cortex Moutan, Lignum Aquilariae Resinatum, Radix Angelicae Dahuricae and Bomedlum Synthethicum.

8. The topical preparation of claim 7 wherein the topical mixture comprises a base mixture capable of topical application on the human skin inside of the navel and/or around the navel and one of the herbs selected from the list of herbs including Cortex Moutan, Lignum Aquilariae Resinatum, Radix Angelicae Dahuricae and Bomedlum Synthethicum.

9. The topical preparation of claim 7 wherein the topical mixture comprises a base mixture capable of topical application on the human skin inside of the navel and/or around the navel and two of the herbs selected from the list of herbs including Cortex Moutan, Lignum Aquilariae Resinatum, Radix Angelicae Dahuricae and Bomedlum Synthethicum.

10. The topical preparation of claim 7 wherein the topical mixture comprises a base mixture capable of topical application on the human skin inside of the navel and/or around the navel and three of the herbs selected from the list of herbs including Cortex Moutan, Lignum Aquilariae Resinatum, Radix Angelicae Dahuricaeand Bornedlum Synthethicum.

11. The topical preparation of claim 7 wherein the topical mixture comprises a base mixture capable of topical application on the human skin inside of the navel and/or around the navel and all four of the herbs selected from the list of herbs including Cortex Moutan, Lignum Aquilariae Resinatum, Radix Angelicae Dahuricae and Bornedlum Synthethicum.

12. The topical preparation of claim 7 wherein the topical mixture comprises an herbal ointment.
